SEQUENCE LISTING (10 sequences)

SEQ ID NO: 1 - DNA, 24 bp, artificial
  Primer
tacaggctactcaactggggattg

SEQ ID NO: 2 - DNA, 21 bp, artificial
  Primer
gctgccctttcttggacgatc

SEQ ID NO: 3 - DNA, 24 bp, artificial
  Primer
ggttctcattcggtcctgcttctg

SEQ ID NO: 4 - DNA, 21 bp, artificial
  Primer
cgcttactgtgctgggatggc

SEQ ID NO: 5 - DNA, 23 bp, artificial
  Primer
ggtgagataagaggctgcgtatg

SEQ ID NO: 6 - DNA, 23 bp, artificial
  Primer
atgttgcgtgtgaggagatgttc

SEQ ID NO: 7 - DNA, 34 bp, artificial
  Primer
tttccaagccagccagagggaggagaaggagttc

SEQ ID NO: 8 - DNA, 28 bp, artificial
  Primer
ctcatgtgcaagttccaggaggccagga

SEQ ID NO: 9 - DNA, 20 bp, artificial
  Primer
acctccgtcctgctctactg

SEQ ID NO: 10 - DNA, 24 bp, artificial
  Primer
ctgacctaaccttccacctaatcc

(19) United States
(12) Patent Application Publication
(10) Pub. No.: US 2017/0029895 A1
(43) Pub. Date: Feb. 2, 2017

(54) DIAGNOSTIC KIT AND METHOD TO DIAGNOSE INCONTINENTIA PIGMENTI

(71) Applicants: UNIVERSITA' DEGLI STUDI DELLA BASILICATA, Potenza (IT); CONSIGLIO NAZIONALE DELLE RICERCHE, Roma (IT)

(72) Inventors: Maria Brigida Lioi, Potenza (IT); Matilde Valeria Ursini, Napoli (IT); Francesca Fusco, Napoli (IT); Mariateresa Paciolla, Potenza (IT)

(21) Appl. No.: 15/302,788

(22) PCT Filed: Apr. 10, 2015
(86) PCT No.: PCT/IB2015/052618

§ 371 Date: Oct. 7, 2016

(30) Foreign Application Priority Data
Apr. 10, 2014 (IT) PZ2014A000004

(51) Int. Cl. C12Q 1/68

(52) U.S. Cl. CPC C12Q 1/6883; C12Q 2600/158; C12Q 2600/156; C12Q 2600/16

(57) ABSTRACT

Diagnostic kit to diagnose Incontinentia Pigmenti (IP) (Pigment Incontinence) by means of which to make an analysis of a biological sample of a human organism to detect alterations in the nucleotide sequence of the IP locus. The diagnostic kit comprising primers for Real Time quantitative PCR analyses.

### FIELD OF THE INVENTION

Forms of embodiment of the present disclosure refer to a diagnostic kit and a method to diagnose Incontinentia Pigmenti (IP) (Pigment incontinence) able to identify alterations, in particular recurring and non-recurring genomic rearrangements, in the IP locus.

### BACKGROUND OF THE INVENTION

Incontinentia Pigmenti (IP; OMIM#308300), or Bloch-Sulzberger Syndrome, is a severe neurocutaneous genodermatosis with dominant inheritance linked to sex chromosome X which is lethal in males and which affects females already at a neonatal age.

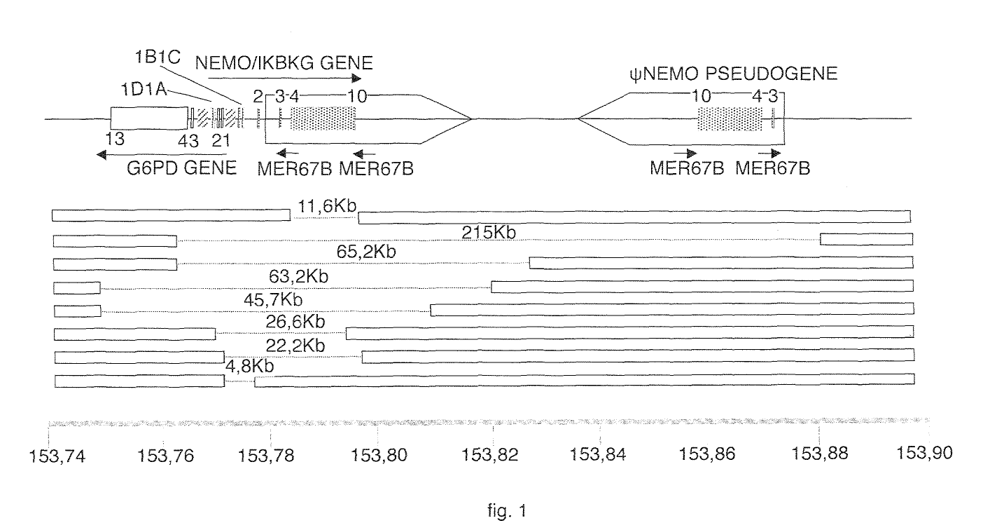

The gene responsible for IP is the gene Inhibitor of Kappa light polypeptide gene enhancer in B-cells, Kinase Gamma (IKBKG), also known as Nuclear Factor-kappaB (NF-kB) Essential MOdulator (NEMO) located in the Xq28 region (see FIG. 1) whose alterations/mutations are responsible for the majority of cases of IP.

IP has been estimated to have a frequency of about 1 in 20,000 births. In affected girls and women, the clinical presentation is highly variable and often severe and it is always associated with specific defects of the epidermis [1, 2]. Further clinical problems in IP patients affect the eyes, teeth, hair, nails and central nervous system, and are present in respectively 26.7%, 36.4%, 27.3%, 12.5% and 22.1% of patients. A high incidence of sporadic cases has also been reported: 3 in 4 patients (76.7%) show no family hereditariness. In these cases the pathology is caused by germinal mosaicism due to the mutation of gene NEMO/IKBKG in one of the two non-affected parents.

The NEMO/IKBKG gene is mutated in 73% of patients [3-6]. The remaining 27% is still without a definitive molecular diagnosis.

The NEMO/IKBKG gene encodes for the regulatory subunit of the Inhibitor of KappaB Kinase (IKK) complex, responsible for the activation of NF-kB, a ubiquitous transcription factor implicated in inflammation, in the immune response, in cell survival, in memory and learning. The heterogeneous clinical presentation, often extremely severe, found in IP is attributed to the role of the NEMO protein which acts as a platform at the point of convergence of the activation of the NF-kB signal [7, 8].

The considerable percentage of patients who do not show mutations in the NEMO/IKBKG encoding sequence has left open the possibility of finding heterogeneous genetic causes for IP.

The classical molecular diagnosis for IP, hitherto performed by analyzing gene NEMO/IKBKG, provides to search for the recurring deletion of exons 4-10 and punctiform mutations by means of PCR and direct sequencing. This analysis, in current conditions, facilitates clinical diagnosis; however, molecular diagnosis in cases where recurring mutations are not present is made difficult by the complexity of the architecture of the IP locus; for this reason 27% of patients lack a conclusive molecular diagnosis.

In particular, some recombination events have been observed, which generate small variations in the number of copies (CNVs) in risk alleleles of the pathology [9, 10]. The presence of highly repeated sequence families of DNA, LCRs and unprocessed pseudogenes in the IP locus can promote mechanisms of homologous recombination and give rise to allelic variants, pathological and not [10]. Moreover, recombinations in the NEMO/IKBKG region, which do not alter the structure of the gene but the number of copies, have been identified in association with cases of haemophilia [11] and X-associated mental retardation [12].

The approach of searching for recurring deletion 4-10 and punctiform mutations by means of PCR and direct sequencing does not allow to identify unknown new rearrangements (pathological and not) in the IP locus. The genomic architecture of said locus is particularly unstable from a genetic point of view as it is frequently affected by deletions or duplications of the gene.

A resolutive therapy for IP does not exist at the moment and therefore an early identification of genetic alterations in the NEMO/IKBKG locus, optimizing the times and costs of analysis, constitutes fundamental information not only to confirm the clinical diagnosis, but above all to start a course of treatment/rehabilitation that is appropriate, personalized and prompt for the patient.

It is in fact essential that the molecular diagnosis for the clinical suspicion of IP is confirmed as soon as the cutaneous lesions start to appear, in order to recruit the patients for clinical follow-up protocols that are effective in preventing the onset of more serious inflammation processes and to apply a protocol of personalized psychomotor rehabilitation.

For example, document US-A-2003/0032055 is known, which describes the diagnosis and treatment of medical conditions associated with a defect in the activation of NFKappaB (NF-KappaB), including IP. However, US-A-2003/0032055 adopts a traditional PCR technique and also provides to identify only one type of genomic alteration. It must be noted that US-A-2003/0032055 describes more than one method of molecular diagnosis by means of techniques that are now obsolete in scientific research, because, in addition to being lengthy and procedurally complicated, they provide to use mutagenic and carcinogenic substances, harmful to the health of the operator: radioactivity and polyacrylamide. Moreover, these methods of molecular diagnosis are only useful for patients who are carriers of punctiform mutations in single exons: i) SSCP, which is based on the ability of punctiform mutations to alter the mobility of small fragments of one-stranded DNA on electrophoretic display on polycarylamide gel under conditions that are not denaturing; and ii) Southern blot, whereby RNA is extracted from the patients' cell lines and converted to cDNA in a retrotranscription reaction (RT-PCR) and is subsequently subjected to hybridization with a radioactively marked probe.

In addition, the only method of diagnosis described in US-A-2003/0032055 that allows to identify more complex mutations is based on experiments with standard PCR starting from patients' genomic DNA and by means of which it is only possible to identify the recurring deletion of exons 4-10 of the NEMO gene but not the duplication of said gene, identified for the first time by Fusco et al. [6]. Although US-A-2003/0032055 attempts to diagnose Incontinentia Pigmenti, moreover according to lengthy, laborious and currently obsolete methods, it has not been able to provide a molecular diagnosis for all patients affected by Incontinentia Pigmenti, but only for those patients who are carriers of a common deletion of exons 4-10 of the NEMO gene and of punctiform mutations in the individual exons. Hence the need to develop a diagnostic method that is rapid and easy to apply in order to identify not only the recurring deletion but also new genomic rearrangements that play a role in IP pathogenesis as described by Fusco et al. [9].

Moreover, at the time application US-A-2003/0032055 was filed, the genomic architecture of the chromosomic region Xq28 was not known to the scientific world, as the complete draft of the human genome had only been made public a few years earlier (Human Genome Project, 2000). The complexity of the genomic architecture of the IP locus and the identification of non-recurring deletions of varying extension in said locus were later described by Fusco et al. [9] and Conte et al. [10] (see FIG. 1).

There is therefore a need to perfect a diagnostic kit and a method for diagnosing IP that can overcome at least one of the disadvantages of the state of the art.

It has thus become necessary to develop a new diagnostic test that allows to identify the genetic/molecular defect also in those cases lacking molecular diagnosis, that is, lacking a conclusive molecular diagnosis.

In these cases, the new diagnostic test thus developed can have diagnostic purposes for non-recurring quantitative alterations in the IP locus and causing other genetically associated pathologies.

Moreover, the new diagnostic test thus developed can constitute a valid analysis tool, as it will standardize and simplify the molecular diagnosis procedure for IP for large-scale application.

The Applicant has devised, tested and embodied the present invention to overcome the shortcomings of the state of the art and to obtain these and other purposes and advantages.

Unless otherwise defined, all the technical and scientific terms used here and hereafter have the same meaning as commonly understood by a person with ordinary experience in the field of the art to which the present invention belongs. Even if methods and materials similar or equivalent to those described here can be used in practice and in the trials of the present invention, the methods and materials are described hereafter as an example. In the event of conflict, the present application shall prevail, including its definitions. The materials, methods and examples have a purely illustrative purpose and shall not be understood restrictively.

### SUMMARY OF THE INVENTION

The present invention is set forth and characterized in the independent claims, while the dependent claims describe other characteristics of the invention or variants to the main inventive idea.

In accordance with the above purpose, forms of embodiment described here, which overcome the limits of the state of the art and eliminate the defects present therein, concern a diagnostic kit for diagnosing Incontinentia Pigmenti by means of which to make an analysis of a biological sample of a human organism to detect alterations in the nucleotide sequence of the IP locus in a human organism, said kit comprising at least two primers for Real time quantitative PCR analysis.

In accordance with forms of embodiment of the present disclosure, the primers are designed for:

i) searching for the recurring pathological deletion 4-10;
ii) searching for the duplication 4-10 of the NEMO/IKBKG gene which constitutes a risk allele for IP;
iii) identifying non-recurring deletions;
iv) controlling a normal genotypic state.

In accordance with forms of embodiment of the present disclosure, the diagnostic kit comprises at least two primers selected from the group that consists of: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10.

In accordance with forms of embodiment of the present disclosure, the diagnostic kit comprises a primer SEQ ID NO 1, a primer SEQ ID NO 2, a primer SEQ ID NO 3, a primer SEQ ID NO 4, a primer SEQ ID NO 5, a primer SEQ ID NO 6, a primer SEQ ID NO 7, a primer SEQ ID NO 8, a primer SEQ ID NO 9 and a primer SEQ ID NO 10.

In accordance with further forms of embodiment described here, a method is provided to diagnose Incontinentia Pigmenti in a human organism, which comprises:

making available a biological sample from a human organism;

analyzing the biological sample to detect alterations in the nucleotide sequence of IP locus, in which the analysis is made by means of Real Time quantitative PCR.

In accordance with forms of embodiment of the present disclosure, the alterations are selected from a group consisting of recurring deletion, non-recurring deletion, duplication/triplication.

In accordance with forms of embodiment of the present disclosure, the alterations are localized in a genomic region containing one or more of either regulation zones, exons, introns, an initiator codon, a stop codon, exon/intron joints, a non-translated region 5′, a non-translated region 3′ of the NEMO/IKBKG gene, or combinations thereof.

In accordance with forms of embodiment of the present disclosure, the Real Time quantitative PCR analysis uses primers designed for:

i) searching for the recurring pathological deletion 4-10;
ii) searching for the 4-10 duplication of the NEMO/IKBKG gene which constitutes a risk allele for IP;
iii) identifying non-recurring deletions;
iv) controlling a normal genotypic state.

In accordance with forms of embodiment of the present disclosure, the Real Time quantitative PCR analysis uses at least one primer selected from the group that consists of: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10.

In accordance with forms of embodiment of the present disclosure, the Real Time quantitative PCR analysis uses at least two primers selected from the group that consists of: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10.

In accordance with forms of embodiment of the present disclosure, the Real Time quantitative PCR analysis uses a primer SEQ ID NO 1, a primer SEQ ID NO 2, a primer SEQ ID NO 3, a primer SEQ ID NO 4, a primer SEQ ID NO 5, a primer SEQ ID NO 6, a primer SEQ ID NO 7, a primer SEQ ID NO 8, a primer SEQ ID NO 9 and a primer SEQ ID NO 10.

These and other aspects, characteristics and advantages of the present disclosure will be better understood with reference to the following description, drawings and attached claims.

The various aspects and characteristics described in the present description can be applied individually where possible. These individual aspects, for example aspects and characteristics described in the attached dependent claims, can be the object of divisional applications.

It is understood that any aspect or characteristic that is discovered, during the patenting process, to be already known, shall not be claimed and shall be the object of a disclaimer.

### DETAILED DESCRIPTION OF A FORM OF EMBODIMENT

We shall now refer in detail to the various forms of embodiment of the present invention, of which one or more examples are given below. Each example is supplied by way of illustration of the invention and shall not be understood as a limitation thereof. For example, the characteristics shown or described insomuch as they are part of one form of embodiment can be adopted on, or in association with, other forms of embodiment to produce another form of embodiment. It is understood that the present invention shall include all such modifications and variants.

Forms of embodiment described here refer to the development of a new diagnostic kit for the disease Incontinentia Pigmenti, able to detect possible alterations in the IP locus.

Forms of embodiment described here provide a diagnostic kit to diagnose IP, the kit comprising five pairs of oligonucleotides or primers, obtained through rational design based on the detailed study of the complex genomic architecture of the IP locus.

The present inventors have conducted extensive research and experiments in the development of new strategies and the use of the latest technologies, such as Real time quantitative PCR, to reveal unknown genomic alterations in those cases of IP that lacked molecular diagnosis.

Forms of embodiment in accordance with the present disclosure therefore refer in particular to:

a method to diagnose IP: a method to detect a pathological condition associated with the IP locus in a human organism, comprising different steps: obtaining or making available a biological sample from the human organism and analyzing the sample to search for alterations in the nucleotide sequence of IP locus;

a diagnostic kit for IP: a diagnostic kit to detect alterations in the nucleotide sequence in the IP locus, the kit comprising five specific pairs of oligonucleotides or primers: two pairs of oligonucleotides used to search for the pathological deletion 4-10 and to search for the duplication 4-10 of the gene constituting a risk allele for the pathology; two pairs of oligonucleotides to detect non-recurring deletions and a further pair to control a normal genotypic state.

The inventive idea in accordance with the present disclosure is therefore based on the use of five pairs of oligonucleotides, or primers—whose sequences are hereby provided as SEQ ID NO 1, SEQ ID NO 2, SEQ ID NO 3, SEQ ID NO 4, SEQ ID NO 5, SNO 6, SEQ ID NO 7, SEQ ID NO 8, SEQ ID NO 9, SEQ ID NO 10, of which two pairs are respectively configured to search for the pathological recurring deletion 4-10 and to search for the duplication 4-10 of the gene (NEMO/IKBKG) that constitutes a risk allele for the pathology, (SEQ ID NO 3, SEQ ID NO 4 and SEQ ID NO 5, SEQ ID NO 6), two pairs to identify non-recurring deletions (SEQ ID NO 7, SEQ ID NO 8 and SEQ ID NO 9, SEQ ID NO 10), and one pair to control a normal genotypic state (SEQ ID NO 1, SEQ ID NO 2).

In accordance with forms of embodiment of the diagnostic method described here, the five pairs of oligonucleotides are used to obtain a reaction mixture which is distributed into wells in conjunction with the DNA sample to be amplified, and then all is subjected to an amplification reaction (Real time quantitative PCR).

The Applicant has designed and configured the oligonucleotides, or primers, so that they are suitable, on the whole, to serve all the following functions:

i) searching for the recurring pathological deletion 4-10;
ii) searching for the 4-10 duplication of the NEMO/IKBKG gene which constitutes a risk allele for the pathology;
iii) identifying non-recurring deletions;
iv) controlling a normal genotypic state.

Table 1 below lists the nucleotide sequences SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, of the oligonucleotides used as forward oligonucleotides and reverse oligonucleotides and which act as amplification primers in Real time quantitative PCR, for the kit and method in accordance with the present disclosure.

TABLE 1

| Name of | Forward | Reverse |
|---|---|---|
| oligonucleotide | oligonucleotide | oligonucleotide |
| Oligo_control | SEQ ID NO 1: | SEQ ID NO 2: |
| | TACAGGCTACTCAAC | GCTGCCCTTTCTTGGAC |
| | TGGGGATTG | GATC |
| Oligo1_del/dup | SEQ ID NO 3: | SEQ ID NO 4: |
| | GGTTCTCATTCGGTC | CGCTTACTGTGCTGGGA |
| | CTGCTTCTG | TGGC |
| Oligo2_del/dup | SEQ ID NO 5: | SEQ ID NO 6: |
| | GGTGAGATAAGAGG | ATGTTGCGTGTGAGGAG |
| | CTGCGTATG | ATGTTC |
| Oligo1_rg | SEQ ID NO 7: | SEQ ID NO 8: |
| | TTTCCAAGCCAGCCA | CTCATGTGCAAGTTCCA |
| | GAGGGAGGAGAAGG | GGAGGCCAGGA |
| | AGTTC | |
| 0ligo2_rg | SEQ ID NO 9: | SEQ ID NO 10: |
| | ACCTCCGTCCTGCTC | CTGACCTAACCTTCCAC |
| | TACTG | CTAATCC |

Forms of embodiment in accordance with the present disclosure allow to identify different types of genomic alteration in the IP locus, also in those patients who do not currently have a molecular response, by means of a protocol that is completely different from the one hitherto used.

The design and development of the oligonucleotides, or primers, by the present inventors originated from the fact that they identified, for the first time in the state of the art, as well as the common deletion of exons 4-10 of the NEMO/IKBKG gene, other non-recurring deletions with a variable extension in the IP locus in Xq28.

The present inventors have conducted a statistical and bioinformatical analysis of the genomic architecture surrounding the breakpoint regions which has revealed a non-random “cluster” of most of the breakpoints inside the NEMO/IKBKG locus. Preliminary experimental data obtained by the inventors show complex junction regions which are the main cause of the genetic duplications/triplications responsible for genomic instability due to the presence of numerous and different repeated/repetitive sequences in both directions. The present inventors, once they had identified the breakpoint cluster, were able to perform a more accurate molecular diagnosis on the DNA of IP patients for whom the genetic cause was unknown, by using the kit in accordance with the present disclosure.

The construction of oligonucleotides suitable to identify CNVs in the NEMO/IKBKG locus has required a complex and detailed study of the genomic organization, combined with a procedure to design the oligonucleotides using bioinformatical instruments.

In particular, the present inventors had to apply great ability and creative thought in the use of said bioinformatical instruments, to exclude zones that were prohibitive for quantitative analysis (highly repeated DNA) and to design the best possible pairs of oligonucleotides, or primers, for a Real time quantitative PCR test on the remaining single copy sequences. The complexity of the procedure was increased, moreover, by the presence of very few single copy regions on which to design the appropriate oligonucleotides for an accurate and extremely specific molecular diagnosis.

The diagnosis has been made, therefore, no longer with the conventional methodology based on PCR amplification, but, in a highly innovative way and new in the state of the art, by means of a Real time quantitative PCR investigation that has made it possible to identify further non-recurring rearrangements, not yet described in literature and until now defying classical analysis.

Although Real time quantitative PCR is now commonly used in research laboratories, recent studies by the present inventors have shown that it is the only technique able to display CNVs with dimensions that are neither big enough to use cytogenetic methods, nor small enough to use simple amplification reactions through PCR. Moreover, the latest data available from the present inventors show that not even advanced latest-generation techniques, such as deep sequencing, allow to reveal said CNVs.

Therefore, the present inventors, in developing the diagnostic kit, and in particular the five pairs of oligonucleotides comprised therein, and the corresponding method to diagnose IP, have inventively overcome considerable complexities connected to:

i) the construction of oligonucleotides in a region of the X chromosome which is difficult to investigate,
ii) acquired knowledge about the identification of the most recombinogenic zones of the locus,
iii) the use of a quantitative method of analysis as the only one able to detect recurring and non-recurring alterations in the region due to an increase or decrease in the number of copies of the locus.

Compared to what is described for example in document US-A-2003/0032055, the nucleotide sequences used in the kit and the method of the present disclosure are new and inventive. Moreover, compared to US-A-2003/0032055, the method used by the present disclosure is different in that it provides a nucleotide analysis by means of Real time quantitative PCR and not by PCR, and a completely different analysis protocol. Furthermore, the present disclosure also differs from US-A-2003/0032055 in the expected results since, while the kit described in US-A-2003/0032055 allows to identify only one type of genomic alteration, the present disclosure allows to identify different types of genomic alteration.

The kit and method according to the present disclosure are therefore based on said five pairs of oligonucleotides, specific for quantitative PCR technology (Real time quantitative PCR), able to determine the number of copies of small informative regions of the IP locus and to detect the presence of deletions or duplications.

From experimental results obtained by the present inventors it emerges that the diagnostic kit and the method according to the present disclosure can constitute a valid non-invasive tool for early diagnosis.

In control-case studies conducted by the present inventors it has been demonstrated that quantitative genomic analysis (determination of the number of copies) makes it possible to identify recurring and non-recurring genomic rearrangements in the IP locus in about 90% of patients.

Considering, moreover, that recombinations in the NEMO/IKBKG region, which do not alter the structure of the gene but the number of copies, have been identified in association with cases of hemophilia and X-associated mental retardation, it can be maintained that the kit and method according to the present disclosure can serve diagnostic purposes for non-recurring quantitative alterations in the IP locus that cause other genetically associated pathologies.

Advantageously, the molecular test conducted on the DNA of patients affected by IP according to the present disclosure, guarantees the analysis of the quantitative genomic profile of the NEMO/IKBKG locus.

By means of the result of the quantitative analysis of this locus according to the present disclosure, clinicians are able to establish whether the subject has the genetic defect causing the pathology and/or whether there is a predisposition due to the presence of risk alleles in the parents, making a predictive diagnosis.

Moreover, the development and marketing of a diagnostic kit and method in accordance with the present disclosure in the field of both molecular and predictive diagnosis constitute not only a fundamental objective for early diagnosis and for choosing adequate and personalized therapies for IP, but also a possibility to transfer the knowledge and basic research technology to industry, and possibly applicable to other pathologies, both rare and less so.

The kit in accordance with the present disclosure has been designed to operate optimally through Real time quantitative PCR technology, with all the commercial MasterMixes according to the protocols of the production company.

It must be emphasized that the present inventors are among the authors of the publications Fusco et al. [9] and Conte et al. [10] and are therefore aware of what is already known in literature and to the scientific world with reference to everything that concerns Incontinentia Pigmenti.

Over the last years, in fact, the research group of the present inventors, attached to the National Research Council (CNR), has become the European Reference Center for the identification of alterations of the NEMO gene found in the DNA of girls and women affected by IP (http://www.orpha.net/consor/cgibin/ClinicalLabs_Search.php?lng=IT&data_id=18677&search=ClinicalLabs_Search_Simple&data_type=Test&title=Diagnosi-molecolare-dell-incontinentia-pigmenti-gene-IKBKG-&MISSING %20CONTENT=Diagnosi-molecolare-dell-incontinentiapigmenti-gene-IKBKG-).

Moreover, during the current year at the Laboratory of Human Molecular Genetics of the CNR in Naples, to which the inventors belong, biological samples have arrived from the USA and Japan with an explicit request to identify unknown alterations in the IP locus, as no molecular response for the illness had been received from any diagnostic center. The need is therefore emphasized for countries outside the EU to have a reference center for the disease, as requested by the Incontinentia Pigmenti International Foundation (IPIF, http://www.ipif.org/) during a meeting with the families of IP patients which took place in Philadelphia (USA) on the 16 Oct. 2014 (http://www.igb.cnr.it/ipgb/events/together-withip-families).

Following the work by Fusco et al. [9] the inventive idea behind the present disclosure was conceived, to develop a diagnostic kit and method, rapid and easy to use, for the identification of both the common rearrangement of the NEMO gene, and also of non-recurring rearrangements which not only affect the NEMO gene, but also alter the adjacent regions, for example the G6PD gene involved in favism, another hereditary genetic disease. The presence of numerous and different repeated/repetitive sequences in the IP locus increases the possibility of genetic recombinations and therefore makes it difficult to identify genomic alterations of varying extension unequivocally for each patient. The kit in accordance with the present disclosure is a valid analysis tool because it standardizes and simplifies the molecular diagnosis method for Incontinentia Pigmenti for a large-scale application without having to resort to use reagents which are harmful to the operator. All the oligonucleotide sequences described in the background of the invention and the relative diagnostic methods have not been included in the object of the present patent application, which focuses on new sequences of oligonucleotides and their method of use, never disclosed in any publication or, more generally, divulged.

In this respect, the present inventors point out that they are included among the authors of the works published by Fusco et al. [9] and Conte et al. [10] and as such they are well aware of what has been divulged in the scientific world on this matter.

The sequences of oligonucleotides described in the present disclosure have never been published nor divulged by explicit request. Moreover, the oligonucleotides described here have been designed following the publication Fusco et al. [9] and the detailed study of the genomic structure of the Xq28 region included therein, precisely to develop a new and inventive kit that could provide genetic information on the IP locus, rapidly and unequivocally, concerning recurring and non-recurring rearrangements.

In addition, we wish to clarify that, while the Real time PCR assay is a known technique in molecular biology and used in different scientific applications, the method described and claimed here, along with a specific experimental protocol, is the only one that allows to identify non-recurring rearrangements in the IP locus which, unlike other genomic regions, cannot be displayed by cytogenetic methods (CGH arrays, chromosome banding, etc.) because the sizes are not big enough, nor by molecular genetic methods (Next Generation Sequencing, Standard PCR, Southern blot, etc.) because the sizes are not small enough.

Application US-A-2003/0032055, as described above, concerns an “incomplete” diagnosis method due to current scientific knowledge, and provides to use numerous molecular biology techniques which are lengthy, complex in application and harmful due to the reagents used, if we consider the health-care and pharmaceutical context in which the speed of method, the application to a large cohort of patients and the health of the operator are of fundamental importance. Moreover, US-A-2003/0032055 concerns sequences of oligonucleotides and a method of diagnosis that are completely different both in purpose and in the instruments from what we have described and claimed.

The application US-A-2003/0032055 provides a kit to identify an alteration of the nucleotide sequence of the NEMO gene comprised between two oligonucleotides selected from a larger group. The kit described in US-A-2003/0032055 provides to use a standard PCR technique and can “only” identify the deletion of exons 4-10 of the NEMO gene. The analysis therefore excludes: i) the duplication of the entire gene, identified for the first time by Fusco et al. [6], in which the large size of the amplicon makes it impossible to display it with the pair of oligonucleotides used by US-A-2003/0032055, and ii) extensive rearrangements, such as those identified by Fusco et al. [9] and Conte et al. [10], covering regions outside the gene analyzed. In view of these considerations, the present inventors maintain, on the other hand, that the new kit described and claimed here, inventively allows a “quick and complete” diagnosis of the entire IP locus.

The present inventors emphasize that the oligonucleotides used in the kit of US-A-2003/0032055 have already been tested by the present inventors and “do not work” for Real time PCR. The oligonucleotides designed for Real time PCR experiments must respect certain conditions that are not always required for simple PCR, and in particular: the amplicon sequence must be unique in the genome and the oligonucleotides must not form homo- and heterodimers. It is worth remembering that the IP locus is a genomic region which is rich in repeated sequences and that the construction of the oligonucleotides suitable to detect alterations in the number of copies requires a detailed study of the genomic organization which, with regard to IP, has been explored only by the present inventors and disclosed by Fusco et al. [9]: all this has required great ability in the use of bioinformatical instruments in order to exclude zones which are prohibitive for quantitative analysis (highly repeated DNA) and to design the best possible pairs of oligonucleotides for a Real time PCR test in the remaining single copy sequences. The complexity of this procedure increases due to the presence of very few single copy regions on which to design the oligonucleotides suitable for an accurate and extremely specific molecular diagnosis. Document US-A-2003/0032055 does not take into account the real genomic architecture of the IP locus and all the pathogenic alterations associated with it, as such information has been made available to the scientific world only in more recent years; it only takes into account the genetic structure (exon/intron) and therefore excludes alterations in the number of copies in regions adjacent to the NEMO gene.

In this context it must be underlined that the kit in accordance with the present description does not refer to alterations in the nucleotide sequence of the “NEMO gene” but to alterations in the nucleotide sequence of the “IP locus”, that is, also those regions adjacent to the gene. In particular, the kit in accordance with the present description provides to identify non-recurring deletions, that is, those pathogenic alterations covering genomic regions which are more extensive compared with the NEMO gene.

Furthermore, it must be underlined that the use of the Real time PCR technique in the method to diagnose Incontinentia Pigmenti in a human organism in accordance with the present description is not a mere choice of a common technique to analyze nucleic acids, as the analysis of a biological sample coming from a human organism provides to identify alterations in the nucleotide sequence of the entire IP locus and not only of the NEMO gene as described by US-A-2003/0032055.

Fusco et al. [6, 9] and Conte et al. [10] identified new pathogenic alterations and new risk alleles for Incontinentia Pigmenti that were not known at the time of US-A-2003/0032055. The new kit according to the present description allows to provide a complete molecular diagnosis, rapid and unequivocal, using a molecular biology technique that is the only one that allows to identify non-recurring rearrangements in the IP locus which, unlike other genomic regions, cannot be displayed by cytogenetic methods (CGH arrays, chromosome banding, etc.) because the sizes are not big enough, nor by molecular genetic methods (Next Generation Sequencing, Standard PCR, Southern blot, etc.) because the sizes are not small enough.

Therefore, the diagnostic method in accordance with the present description is advantageously able to identify alterations of the nucleotide sequence “of the entire IP locus” using the Real time PCR technique, according to a specific protocol perfected for the purpose, as the Real time PCR technique is the only one able to identify complex rearrangements in this genomic region.

The present inventors once again state that the kit described in document US-A-2003/0032055 provides to identify punctiform mutations using techniques that are now obsolete and harmful to the operator (SSCP, Southern blot) and, with regard to more complex genomic alterations, the use of the standard PCR technique and of oligonucleotides that allow “only” to identify the deletion of exons 4-10 of the NEMO gene, excluding from the analysis: i) the duplication of the entire gene, identified for the first time by Fusco et al. [6], in which the large size of the amplicon cannot be displayed with the pair of oligonucleotides used in US-A-2003/0032055, and ii) extensive deletions, such as those identified by Fusco et al. [9], covering regions outside the gene analyzed, and therefore outside the genic region examined in document US-A-2003/0032055.

We must point out that the present inventors are the authors of the publications relating to the discovery of new genomic rearrangements in the IP locus and of risk alleles for the disease (Fusco et al. [6, 9]; Conte et al. [10]).

We must also point out that the present inventors constitute the National and International Reference Center for Incontinentia Pigmenti (http://www.orpha.net/consor/cgibin/ClinicalLabs_Search.php?lng=IT&data_id=18677&search=ClinicalLabs_Search_Simple&data_type=Test&title=Diagnosi-molecolare-dell-incontinentia-pigmenti-gene-IKBKG-&MISSING %20CONTENT=Diagnosi-molecolare-dell-incontinentiapigmenti-gene-IKBKG-).

We must also point out that the present inventors collaborate with some of the inventors of application US-A-2003/0032055 as the scientific literature shows and, as such, they are aware of any kit and/or method claimed by them.

We must also point out that the present inventors are among the most competent scholars in the subject of Incontinentia Pigmenti in the scientific world as scientific literature shows.

Consequently, the kit and method in accordance with the present description, originating from the expertise acquired over the years by the present inventors, which are able to identify recurring and non-recurring rearrangements in the entire IP locus, allow to provide patients, in an advantageous way unobtainable with known techniques, with a “quick and unequivocal” molecular diagnosis for the presence of alterations in the number of copies of the genomic region comprising the NEMO gene and adjacent zones, also for those patients who were previously undiagnosed.

With regard to the function of the oligonucleotides described here of identifying new deletions, a general overview of non-recurring deletions is given in FIG. 1 and concerns deletions known in literature (Fusco et al. [9]; Conte et al. [10]) and identified using a Real time PCR technique.

We stress that it is not possible to trace the origin of the sequence of oligonucleotides of the kit and method described here from the information contained in the documents Fusco et al. [9] and Conte et al. [10] as these are new oligonucleotides designed following the discovery of the new rearrangements present in the IP locus. Documents Fusco et al. [9] and Conte et al. [10] have the sole purpose of describing the discovery of new genomic rearrangements in the IP locus without discussing the specific protocol used for the Real time PCR experiments nor the sequences of oligonucleotides used. In order to identify new deletions in a rapid and unequivocal way, the present inventors have designed further oligonucleotides able to highlight in a single Real time PCR reaction the presence of genomic alterations, in a different manner from described in document Fusco et al. [9] which provides a greater number of amplification reactions. With the kit and method in accordance with the present description, it is possible to provide precise information regarding the presence/absence of alterations in the number of genomic copies of the IP locus in a short time and at low cost.

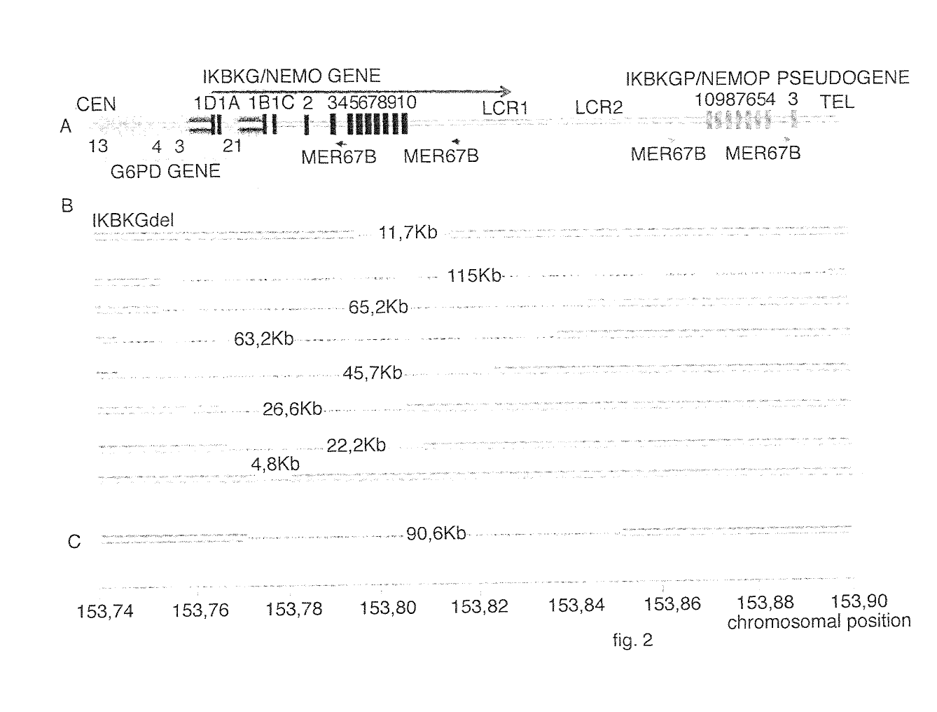

In the field there is a long-felt and never satisfied need to provide a diagnostic kit and method able to overcome the disadvantages of the state of the art; this is witnessed by the fact that other persons of skill (medical doctors, researchers, geneticists) have not been able to design oligonucleotides in the IP locus using the information given in documents Fusco et al. [9] and Conte et al. [10]: the present inventors have received numerous requests for molecular diagnosis of IP from different countries (France, Japan and USA). In particular, it is precisely on a DNA sample coming from the USA that a new genomic deletion was identified using the kit described here, as can be seen in zone C of FIG. 2 where:

A) Genomic Structure of the IP locus in the Xq28 region.
B) General overview of the recurring (IKBKGdel) and non-recurring rearrangements in the IP locus published in Fusco et al. [9] and mentioned in Conte et al. [10].
C) New non-recurring rearrangement (previously unpublished and undisclosed data) identified by the kit and method according to the present description.

In view of the above, the present inventors believe that the oligonucleotides described here cannot be designed with the usual design knowledge of oligonucleotides in the state of the art, since a detailed study of the genomic organization of the IP locus is necessary: all this has required from the present inventors a great ability in the use of bioinformatical tools to exclude zones which are prohibitive for quantitative analysis (highly repeated DNA) and to design the best possible pairs of oligonucleotides for a Real time PCR test in the remaining one copy sequences. The complexity of the procedure is increased, moreover, by the presence of very few single copy regions on which to design the oligonucleotides suitable for an accurate and extremely specific molecular diagnosis.

The molecular diagnosis that uses the kit and method in accordance with the present description has made it possible to identify, therefore, further non-recurring rearrangements, not yet disclosed. Although Real time PCR has now become commonly used in research laboratories, recent studies by the present inventors prove that it is the only technique able to display variations in the number of genomic copies in the IP locus with dimensions that are neither big enough to use cytogenetic methods, nor small enough to use simple PCR amplification reactions. Moreover, the latest experimental data available from the research laboratory of the present inventors show that not even advanced and latest-generation techniques, such as deep sequencing, allow to reveal the genomic alterations.

We therefore believe that the diagnostic kit and method described here bring advantages that were not conceivable before and obtainable from the state of the art and referring to:

i) the construction of oligonucleotides in a region of the X chromosome which is difficult to investigate;
ii) acquired knowledge about the identification of the most recombinogenic zones of the locus,
iii) the use of quantitative analysis methods as the only method able to detect recurring and non-recurring alterations in the region due to an increase or decrease in the number of copies of the locus.

### EXAMPLE

For the preparation of the diagnostic kit for IP in accordance with forms of embodiment described here, the lyophilized components, that is, the five pairs of oligonucleotides SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, are resuspended in milli-Q sterile water and stored at −20° C. In optimal reaction conditions, the efficiency of the amplification is >95%.

The procedure provides to prepare a reaction mixture as described in Table 2:

TABLE 2

| Components | Concentrations | |
|---|---|---|
| Genomic DNA | 25 | ng |
| SYBR Green MasterMix | 1X | |
| Forward oligo | 200 | nM |
| Reverse oligo | 200 | nM |
| RNAse/DNAse free water | | |

The final volume of each well is 20 μl.

The temperature program will have to be set in accordance with the instructions of the Real time PCR instrument in use, maintaining an annealing temperature equal to 60° C.

It is clear that modifications and/or additions of parts can be made to the diagnostic kit and method for diagnosing Incontinentia Pigmenti as described heretofore, without departing from the field and scope of the present invention.

It is also clear that, although the present invention has been described with reference to some specific examples, a person of skill in the art shall certainly be able to achieve many other equivalent forms of diagnostic kit and method for diagnosing Incontinentia Pigmenti, having the characteristics as set forth in the claims and hence all coming within the field of protection defined thereby.

Although the above description refers to forms of embodiment of the invention, other and further forms of embodiment can be provided without for this reason departing from the main field of protection, the field of protection is defined according to the claims below.

### BIBLIOGRAPHICAL REFERENCES

[1] Fusco F, Pescatore A, Steffann J, Royer G, Bonnefont J P, Ursini M V. Clinical Utility Gene Card for: Incontinentia Pigmenti. Eur J Hum Genet. 2012 Oct. 10. doi: 10.1038/ejhg.2012.227.
[2] Scheuerle A, Ursini M V. Incontinentia Pigmenti. In: Pagon R A, Bird T D, Dolan C R, Stephens K, Adam M P, editors. GeneReviews™ [Internet]. Seattle (Wash.): University of Washington, Seattle; 1993-1999 Jun. 8 [updated 2010 Oct. 28].
[3] F. Fusco, T. Bardaro, G. Fimiani, V. Mercadante, M. G. Miano, G. Falco, A. Israel, G. Courtois, M. D'Urso and M. V. Ursini. Molecular analysis of the genetic defect in a large cohort of IP patients and identification of novel NEMO mutations interfering with NF-kB activation. Human Molecular Genetics, 2004; 13: 1763-1773;
[4] Fusco F., Fimiani G., Tadini G., D'Urso M., Ursini M. V. Clinical diagnosis of Incontinentia Pigmenti in a cohort of male patients. J Am Acad Dermatol, 2007; 56: 264-267;
[5] Fusco F., Pescatore A., Bal E., Ghoul A., Paciolla M., Lioi M B., D'Urso M., Hadj Rabia S., Bodemer C., Bonnefont J P., Munnich A., Miano M G., Smahi A. and Ursini M V. Alterations of the ikbkg locus and diseases: an update and a report of 13 novel mutations. Human Mutation, 2008; 29: 595-604;
[6] Fusco F., Paciolla M., Pescatore A., Lioi M B., Ayuso C., Faravelli F., Gentile M., Zollino M., D'Urso M., Miano M G., and Ursini M V. Microdeletion/duplication at the Xq28 IP Locus causes de novo IKBKG/NEMO/IKKgamma exon4_10 deletion in Families with Incontinentia Pigmenti. Human Mutation, 2009; 30:1284-91;
[7] Sebban-Benin H, Pescatore A, Fusco F, Pascuale V, Gautheron J, Yamaoka S, Moncla A, Ursini M V, Courtois G. Identification of TRAF6-dependent NEMOpolyubiquitination sites through analysis of a new NEMO mutation causino Incontinentia Pigmenti. Hum Mol Genet. 2007 Dec. 1; 16(23):2805-15. Epub 2007 Aug. 29. Erratum in: Hum Mol Genet. 2008 Feb. 14; 17(4):639;
[8] Gautheron J, Pescatore A, Fusco F, Esposito E, Yamaoka S, Agou F, Ursini M V, Courtois G. Identification of a new NEMO/TRAF6 interface affected in Incontinentia Pigmenti pathology. Hum Mol Genet. 2010; 19:3138-49;
[9] Fusco F, Paciolla M, Napolitano F, Pescatore A, D'Addario I, Bal E, Lioi M B, Smahi A, Miano M G, Ursini M V. Genomic architecture at the Incontinentia Pigmenti locus favours de novo pathological alleles through different mechanisms. Hum Mol Genet. 2012 Mar. 15; 21(6):1260-71. doi: 10.1093/hmg/ddr556;
[10] Conte M I, Pescatore A, Paciolla M, Esposito E, Miano M G, Lioi M B, McAleer M A, Giardino G, Pignata C, Irvine A D, Scheuerle A E, Royer G, Hadj-Rabia S, Bodemer C, Bonnefont J P, Munnich A, Smahi A, Steffann J, Fusco F, Ursini M V. Insight into IKBKG/NEMO locus: report of new mutations and complex genomic rearrangements leading to incontinentia pigmenti disease. Hum Mutat. 2014 February; 35(2):165-77. doi: 10.1002/humu.22483.

1. Diagnostic kit to diagnose Incontinentia Pigmenti (IP) (Pigment Incontinence) by means of which to make an analysis of a biological sample of a human organism to detect alterations in the nucleotide sequence of the IP locus, said diagnostic kit comprising primers for Real Time quantitative PCR analyses.

2. Diagnostic kit as in claim 1, wherein said primers are designed for:
i) searching for the recurring pathological deletion 4-10;
ii) searching for the duplication 4-10 of the NEMO/IKBKG gene which constitutes a risk allele for IP;
iii) identifying non-recurring deletions;
iv) controlling a normal genotypic state.

3. Diagnostic kit as in claim 1, said diagnostic kit comprising at least two primers selected from the group that consists of: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10.

4. Diagnostic kit as in claim 1, said diagnostic kit comprising a primer SEQ ID NO 1, a primer SEQ ID NO 2, a primer SEQ ID NO 3, a primer SEQ ID NO 4, a primer SEQ ID NO 5, a primer SEQ ID NO 6, a primer SEQ ID NO 7, a primer SEQ ID NO 8, a primer SEQ ID NO 9 and a primer SEQ ID NO 10.

5. Method to diagnose Incontinentia Pigmenti (IP) in a human organism, which comprises:
making available a biological sample from a human organism;
analyzing the biological sample to detect alterations in the nucleotide sequence of IP locus, in which the analysis is made by means of Real Time quantitative PCR.

6. Method as in claim 5, wherein said alterations are selected from the group which consists of recurring deletion, non-recurring deletion and duplication/triplication.

7. Method as in claim 6, wherein said alterations are localized in a genomic region containing one or more of either regulation zones, exons, introns, an initiator codon, a stop codon, exon/intron joints, a non-translated region 5′, a non-translated region 3′ of the NEMO/IKBKG gene, or a combination thereof.

8. Method as in claim 6, wherein the Real Time quantitative PCR analysis uses primers designed for:
i) searching for the recurring pathological deletion 4-10;
ii) searching for the 4-10 duplication of the NEMO/IKBKG gene which constitutes a risk allele for IP;
iii) identifying non-recurring deletions;
iv) controlling a normal genotypic state.

9. Method as in claim 6, wherein the Real Time quantitative PCR analysis uses at least one primer selected from the group that consists of: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10.

10. Method as in claim 6, wherein the Real Time quantitative PCR analysis uses at least two primers selected from the group that consists of: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10.

11. Method as in claim 6, wherein the Real Time quantitative PCR analysis uses a primer SEQ ID NO 1, a primer SEQ ID NO 2, a primer SEQ ID NO 3, a primer SEQ ID NO 4, a primer SEQ ID NO 5, a primer SEQ ID NO 6, a primer SEQ ID NO 7, a primer SEQ ID NO 8, a primer SEQ ID NO 9 and a primer SEQ ID NO 10.
